(19) United States
(12) Patent Application Publication
(10) Pub. No.: US 2008/0182985 A1
(43) Pub. Date: Jul. 31, 2008

(54) TYROSINE KINASE INHIBITORS

(76) Inventors: Wenlin Huang, Guangzhou City (CN); Xiaohong Zhou, Guangzhou City (CN)

Correspondence Address: OCCHIUTI ROHLICEK & TSAO, LLP, 10 FAWCETT STREET, CAMBRIDGE, MA, 02138, US

(21) Appl. No.: 11/939,291

(22) Filed: Nov. 13, 2007

Related U.S. Application Data
(63) Continuation-in-part of application No. PCT/CN2005/000661, filed on May 12, 2005.

(51) Int. Cl. C07D 239/94

(52) U.S. Cl. 544/293

(57) ABSTRACT

The present invention relates to quinazoline derivatives represented by general formula (I):

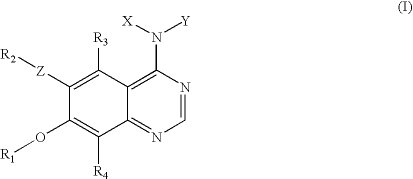

wherein X, Y, Z, R₁, R₂, R₃, and R₄ are as defined herein. The invention also relates to a method of preparing these compounds, and use of these compounds for inhibiting tumor growth.

### CROSS REFERENCE

This application is a continuation-in-part of PCT Application No. PCT/CN2005/000661, filed on May 12, 2005. The contents of the international application are incorporated by reference.

### BACKGROUND

Solid tumors rely on tumor neovascularization in their formation, development, recurrence and metastasis. In other words, vascularization of a tumor is a prerequisite for the growth and metastasis of solid tumors. Hunger therapy of tumor, namely inhibition of tumor neovascularization by cutting off the blood supply to tumor tissue, is deemed as one of the most promising new methods of treatment of solid tumors.

Formation of normal tissue and maintenance of its function rely on the transmembrane signaling cascade from the cytoplasm to the nucleus, which controls the transcription and regulation of genes. Cancer is the result of abnormal cellular activities, such as changes in cell growth, cell survival, and cellular function, and loss of differentiation ability of cell caused by disordered signaling pathway to form a tumor. The development of tumor relies on its host by way of neovascularization to utilize the nutrient and oxygen in the host, during which certain growth factor from tumor stimulates the signaling of host endothelial cell and promotes tumor angiogenesis by extending the existing vessels. The angiogenesis rate in adults is very low, and only the endometrium retains normal angiogenesis activity. For the above reasons, it will be very effective to block the formation of pathogenic vessels by targeting the signaling pathway involved in angiogenesis.

Vascular Endothelial Growth Factor (VEGF) is a growth factor involved in tumor angiogenesis, and plays a role in hormonal regulation of the differentiation of endothelial cell. Solid tumor development is closely related with expression of VEGF. It is shown by current studies that many diseases, including malignant tumors, are associated with angiogenesis (Fan, et al, 1995, Trends Pharmacol. Sci. 16, 57-66; Folkman, 1995, Nature Medicine, 27-31). Change in vasopermeability is thought to have a role in both normal and pathological physiological processes (Cullinan-Bove, et al, 1993, Endocrinology 133, 829-837; Senger, et al, 1993, Cancer and Metastasis Reviews. 12, 303-324), and VEGF is an important stimulating factor in the normal and pathological angiogenesis and change in vasopermeability (Jakeman, et al, 1993, Endocrinology 133, 848-859; Kolch, et al, 1995, Breast Cancer Research and Treatment, 36, 139-155; Connolly, et al, 1989, J. Biol. Chem. 264, 20017-20024). Tumor growth can be inhibited by VEGF antagonism by use of sequestration of VEGF by antibody (Kim, 1993, Nature 362, 841-844).

Improved expression of VEGF is caused by stimulation of multiple factors, such as activation of proto-oncogene and hypoxemia. Hypoxemia of solid tumor may result from the improper perfusion of tumor patient. Besides promotion of neovascularization, VEGF has effect in improvement of vasopermeability, which accelerates the exchange of nutrient and metabolite between tumor and neighboring tissue, and overcomes the barricade of vessel wall to allow metastasis of tumor to distant tissues.

VEGF has tyrosine kinase activity, and can activate the related signaling pathway and promote the tumor neovascularization upon binding with tyrosine kinase as its receptor. Receptor tyrosine kinases (RTKs) activated by the binding of VEGF and its receptor plays an important role in the biochemical signal transduction across cytoplasmic membrane, and can influence growth and metastasis of tumor. The trans-membrane molecule is characterized by that the extracellular ligand binding domain and endocellular tyrosine kinase domain are linked by the fragment in the cytoplasmic membrane. The binding of ligand and receptor stimulates the tyrosine kinase activity associated with receptor, and induces the phosphorylation of tyrosine residue on the receptor and other endocellular molecules. The phosphorylation of tyrosine switches on the signal cascade, and produces multiple cellular responses. Till now at least 19 different RTK subfamilies defined by amino acid sequence homology have been identified, one of which includes Flt (also called Flt1) and Flt4 (both of which are similar to fms), and KDR (also called Flk-1) with kinase domain region. It has been proved that two of the relevant RTKs, Flt and KDR, can bind with VEGF with high affinity (De Vries, et al, 1992, Science 255, 989-991; Terman, et al, 1992, Biochem. Biophys. Res. Comm., 1992, 187, 1579-1586). Binding of VEGF with receptor expressed in heterogenous cell is related with tyrosine phosphorylation level of protein and change in calcium flow.

It is proved by the above mentioned studies that VEGF is specific to neovascularization of solid tumor and is a critical regulating factor directly and positively regulating vascular endothelial cells. VEGF-KDR/Flk-1 pathway becomes one of the major targets in tumor therapy by inhibition of tumor angiogenesis. Inhibition of tyrosine kinase activity is an important way of blocking tumor angiogenesis.

### SUMMARY

The present invention features quinazoline compounds of formula (I):

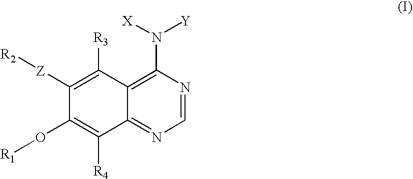

in which

X represents H, methyl, or C₁-C₄ alkyl, preferably —H or methyl, most preferably —H;

Y represents

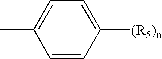

(i.e., substituted phenyl), wherein n is 1, 2, 3, or 4, and R₅, independently, represents H, methyl, trifluoromethyl, nitro, cyano, C₂-C₄ alkyl, C₂-C₄ alkoxyl, N—(C₂-C₄) alkylamino, enzyme, hydroxyl, N,N-triaza(C₁-C₄) alkylamino, C₁-C₄ alkylthio or C₁-C₄ alkylsulfonyl, preferably C₂-C₄ alkyl, nitro, cyano, C₂-C₄ alkoxyl, N—(C₂-C₄) alkylamino, hydroxyl, or C₁-C₄ alkylthio, most preferably C₂-C₄ alkyl, C₂-C₄ alkoxyl, or N—(C₂-C₄) alkylamino;

Z represents

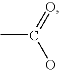

—O, —S or —NH, preferably

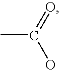

—0, or —S, most preferably

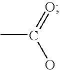

R₁ represents C₁-C₄ alkyl, preferably methyl;

R₂ represents C₁-C₅ alkyl-R₆, C₂-C₆ alkenyl-R₆, or C₂-C₆ alkynyl-R₆, wherein the alkyl, alkenyl, and alkynyl are optionally substituted with one or more alkynyl, enzyme, or amino and R₆ represents 4-piperidyl optionally substituted with one or more alkynyl, enzyme or amino; preferably R₂ represents C₁-C₅ alkyl-R₆ or C₂-C₆ alkenyl-R₆, wherein R₆ represents unsubstituted or substituted 4-piperidyl, optionally with one or more substituents of alkynyl, enzyme or amino on alkyl, alkenyl, alkynyl or 4-piperidyl; most preferably, R₂ represents C₁-C₅ alkyl-R₆, wherein R₆ preferably represents 4-piperidyl, and most preferably represents 4-ethyl-1-piperidyl: and

R₃ and R₄, independently, represent H, methyl, C₁-C₄ alkyl, C₂-C₆ alkenyl, C₂-C₆ alkynyl, cycloalkyl, or heterocycloalkyl, preferably H, methyl, C₁-C₄ alkyl, or C₂-C₆ alkenyl; R₃ is preferably C₁-C₄ alkyl, and most preferably methyl; and R₄ is preferably H.

The term “alkyl” refers to a saturated, linear or branched hydrocarbon moiety, such as —CH₃, —CH(CH₃)₂, or —CH₂—. The term “alkenyl” refers to a linear or branched hydrocarbon moiety that contains at least one double bond, such as —CH═CH—CH₃ or —CH═CH—CH₂—. The term “alkynyl” refers to a linear or branched hydrocarbon moiety that contains at least one triple bond, such as —C≡C—CH₃ or —C≡C—CH₂—. The term “cycloalkyl” refers to a C₃-C₈ saturated, cyclic hydrocarbon moiety, such as cyclohexyl or cyclohexylene. The term “heterocycloalkyl” refers to a C₁-C₈ saturated, cyclic moiety having at least one ring heteroatom (e.g., N, O, or S), such as 4-tetrahydropyranyl or 4-tetrahydropyranylene. The term “alkoxyl” refers to a radical of —O-alkyl. The term “alkylamino” refers to an alkyl-substituted amino group. The term “alkylthio” refers to a radical of —S-alkyl.

Alkyl, alkenyl, alkynyl, cycloalkyl, and heterocycloalkyl, mentioned herein include both substituted and unsubstituted moieties, unless specified otherwise. Possible substituents on cycloalkyl and heterocycloalkyl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, acyl, thioacyl, acyloxy, carboxyl, and carboxylic ester. On the other hand, possible substituents on alkyl, alkenyl, or alkynyl include all of the above-recited substituents except C₁-C₁₀ alkyl.

The term “enzyme” refers to an active protein, which, when attached to a substrate, generates effective physiological reactions.

All of the quinazoline compounds described above include the compounds themselves, as well as their salts. The salts, for example, can be formed between a positively charged moiety (e.g., amine) on the compounds and an anion. Suitable anions include, but are not limited to, chloride, bromide, iodide, sulfate, nitrate, phosphate, citrate, methanesulfonate, trifluoroacetate, and acetate.

This invention also features a method of inhibiting angiogenesis using a compound of formula (I). Further, it features a method of treating tumor using a compound of formula.

Other features or advantages of the present invention will be apparent from the following detailed description of several embodiments and the figures, and also from the appending claims.

### DESCRIPTION OF THE FIGURES

FIG. 1 shows dose dependence of epiphyseal growth plate of rat joint on the inventive compound

FIG. 2 shows inhibitive effect of the inventive compound on human prostate tumor transplanted in nude mouse by use of PC-3

FIG. 3 shows inhibitive effect of the inventive compound on the growth of colon carcinoma cell Lovo

FIG. 4 shows inhibitive effect of the inventive compound on transplanted tumor in the nude mouse model constructed by use of colon carcinoma cell LoVo

### DETAILED DESCRIPTION

The compounds of this invention can be prepared from compound (III) by removing the protective group —P².

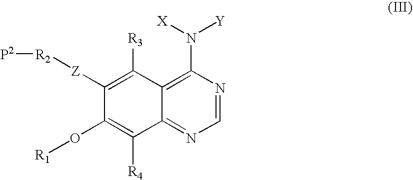

R₁, R₂, R₃, R₄, Z, P², X and Y in compound (III) are described below:

R₁ represents methyl or C₁-C₄ alkyl;

R₂ represents C₁-C₅ alkyl-R₆, C₂-C₆ alkenyl-R₆, or C₂-C₆ alkynyl-R₆, wherein the alkyl, alkenyl, and alkynyl are optionally substituted with one or more alkynyl, enzyme, or amino; and R₆ is 4-piperidyl substituted optionally with one or more alkynyl, enzyme, or amino;

R₃ and R₄, independently, represent —H, methyl, C₁-C₄ alkyl, C₂-C₆ alkenyl, C₂-C₆ alkynyl, cycloalkyl, or isocycloalkyl;

X represents —H, methyl, or C₁-C₄ alkyl;

Y represents

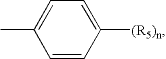

wherein n is 1, 2, 3 or 4, and R₅, independently, is H, methyl, trifluoromethyl, nitro, cyano, C₂-C₄ alkyl, C₂-C₄ alkoxyl, N—(C₂-C₄) alkylamine, enzyme, hydroxyl, N,N-triaza(C₁-C₄) alkylamine, C₁-C₄ alkylthio, or C₁-C₄ alkylsulfonyl;

Z represents —O, —NH,

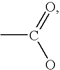

### or —S; and

P² represents a protective group (e.g., carbamate or tert-butoxycarboxyl).

The term “protecting group” is known in the art, for example, as described in Protective Groups in Organic Synthesis (T. W. Greene and R. G. Wuts, 2^{nd} Ed. Wiley 1991); Examples of protective groups include tert-butoxycarboxyl, tert-pentoxycarboxyl, cyclobutoxycarboxyl, propoxycarboxyl, methoxycarboxyl, ethoxycarboxyl, isopropoxycarboxyl, allyloxycarboxyl, and benzyloxycarboxyl.

The reaction is preferably carried out in the presence of acid, including inorganic acid such as HCl or HBr, or organic acid such as trifluoroacetic acid or trifluoromethanesulfonic acid.

The reaction may be carried out in an inert solvent such as dichloromethane or trichloromethane in the presence of a trace amount of water.

A reaction temperature of 10-100° C., preferably 20-80° C., can promote the reaction.

The reaction may produce free base of the compound of this invention or its salt (carrying the acid mentioned above). The salt may be treated by a conventional method to prepare the free base compound.

The compounds of this invention may also be synthesized by known chemical synthesis methods, such as those described in European patent publications 0520722, 0566226, 0602851 and 0635498, and international patent applications WO97/22596, WO97/30035, WO97/32856, and WO98/133541. The methods, as described hereinafter, are deemed as another feature of the present patent. Some of the essential starting materials can be synthesized according to standard procedures of organic chemistry, and their synthetic methods are disclosed in, but not limited to, the embodiments described hereinafter. Other necessary starting materials may be synthesized by methods similar to those described in the manual of organic chemistry.

The compounds of this invention inhibit tyrosine kinase activity, thereby blocking VEGF-induced angiogenesis. As such, these compounds can be used to treat tumor.

Without further elaboration, it is believed that the above description has adequately enabled the present invention. The following specific embodiments are, therefore, to be construed as merely illustrative, and not limitative of the remainder of the disclosure in any way whatsoever. All of the publications cited herein are hereby incorporated by reference in their entirety.

### EXAMPLES

### Example 1

A compound of this invention was prepared by removing the protective group in compound (III)

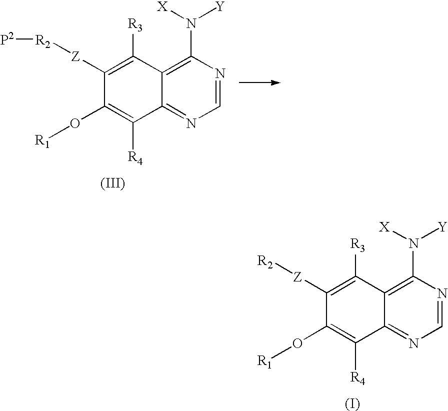

In the above compounds, R₁ is methyl, R₂ is 4-ethylpiperidyl, R₃ is methyl, R₄ is —H,

Z is

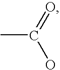

P² is tert-butoxycarboxyl, X is H and Y is methylphenyl.

Compound (III) in 0.1 mol/L HCl solution containing 15% (v/v) of 0.1 mol/L trichloromethane and 0.1% (v/v) of H₂O was stirred and heated in a water bath at 70° C. for 20 min. The reaction mixture was filtered to collect the precipitate, which was dried to afford Compound (I) as a white powder. Compound (I) was water soluble and had a pH value of 6.4.

### Example 2

A compound of this invention was synthesized from compound (III).

In the above compounds, R₁ is ethyl, R₂ is 4-vinylpiperidyl, R₃ is —H, R₄ is methyl, X is methyl, Y is ethylphenyl, Z is

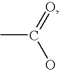

and P² is cyclobutoxycarboxyl.

Compound (III) and a corresponding pyridine compound in 0.2 mol/L HBr solution containing 8% (v/v) of 0.05 mol/L trichloromethane and 0.05% (v/v) of H₂O were stirred and heated in a 50° C. water bath for 30 min. The reaction mixture was filtered to collect the precipitate, which was dried to afford Compound (I) as a white powder. Compound (I) was water soluble and had a pH value of 6.4.

### Example 3

Compound (I) and its salt can be synthesized from compound (III).

In the above compounds, R₁ is methyl, R₂ is 4-ethynylpiperidyl, R₃ is ethyl, R₄ is —H, X is methyl, Y is methylphenyl, Z is —NH, and P² is ethoxycarboxyl.

Compound (III) and a corresponding pyridine compound in 0.1 mol/L trifluoroacetic acid solution containing 15% (v/v) of 0.05 mol/L dichloromethane and 0.2% (v/v) of H₂O were stirred and heated in a 100° C. water bath for 20 min. The reaction mixture was filtered to collect the precipitate, which was dried to afford Compound (I) as a white powder. Compound (I) was water soluble and had a pH value of 6.4.

### Example 4

Compound (I) was synthesized from compound (III).

In the above compounds, R₁ is butyl, R₂ is 4-vinylpiperidyl, R₃ is pentynyl, R₄ is propenyl, X is propyl, Y is nitro, Z is —NH, and P² is benzyloxycarboxyl.

Compound (III) and a corresponding pyridine compound were added in the equal molar amounts into 0.15 mol/L trifluoromethanesulfonic acid solution containing 12% (v/v) of 0.08 mol/L dichloromethane and 0.1% (v/v) of H₂O. The reaction mixture was stirred at 10° C. in a water bath for 60 min and then filtered to collect the precipitate. The precipitate was dried to afford compound (I) as a white powder. Compound (I) was water soluble and had a pH value of 6.4.

### Example 5

Compound (I) was synthesized from compound (III).

In the above compounds, R₁ is propyl, R₂ is 4-vinylpiperidyl, R₃ is —H, R₄ is methyl, X is methyl, Y is ethylphenyl, Z is —S, and P² is allyloxycarboxyl.

Compound (III) and a corresponding pyridine compound were added in the equal molar amounts in 0.08 mol/L HCl solution containing 6% (v/v) of 0.05 mol/L tetrahydrofuran and 0.15% (v/v) of H₂O. The reaction mixture was stirred at a 60° C. water bath of for 30 min and filtered to collect the precipitate, which was dried to afford compound (I) as a white powder. Compound (I) was water soluble and had a pH value of 6.4.

### Example 6

Compound (I) was synthesized from compound (III).

In the above compounds, R₁ is ethyl, R₂ is 4-vinylpiperidyl, R₃ is butenyl, R₄ is methyl, X is methyl, Y is ethylphenyl, Z is

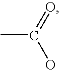

and P² is tert-pentyloxycarboxyl.

Compound (III) and a pyridine compound were added in the equal molar amounts to 0.2 mol/L HCl solution containing 5% (v/v) of 0.15 mol/L N,N-dimethylacetamide and 0.02% (v/v) of H₂O. The reaction mixture was stirred at 80° C. water bath for 30 min, and filtered to collect the precipitate, which was dried to afford compound (I) as a white powder. Compound (I) was water soluble and had a pH value of 6.4.

Pharmacodynamic tests of the following compound are described in embodiments 7 to 13.

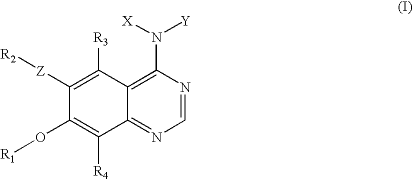

In compound (I), R₁ is methyl, R₂ is 4-ethylpiperidyl, R₃ is methyl, R₄ is —H, X is —H, Y is methylphenyl, and Z is

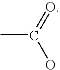

### Example 7

The test compound in sterile distilled water was administered to a 4-8 week old female rat (wostar-derived, Alderley Park) via subcutaneous injection at a dose of 0.25 mg/kg/day over 14 days. The epiphyseal tissue of a leg of the rat was stained with haematoxylin and eosin, and the binding site of the epiphyseal plate was measured for dose-effect analysis. As shown in FIG. 1, overgrowth of the epiphyseal plate resulted in increasing dose-dependence of zona cartilaginea, and when the injection dose was 50 mg/kg/day or 100 mg/kg/day, compound (I) similarly inhibited VEGF signal and inhibited angiogenesis in vivo.

### Example 8

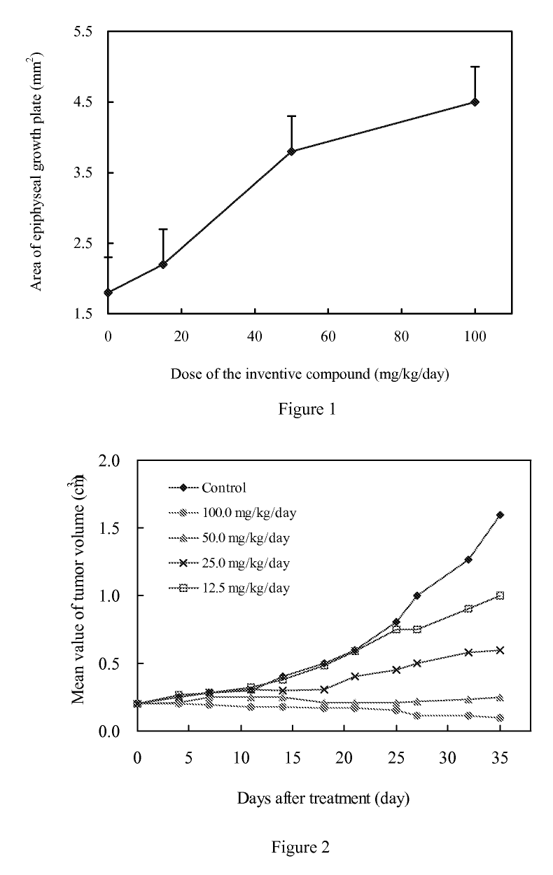

Male nude mice (6 week old) were transplanted with human prostate tumor cell PC-3. After tumor volume reached 0.2 cm³, the mice were randomly divided into five groups. Each group was treated with compound (I) in sterile distilled water via intratumoral injection for 7 days at a dosage of 100 mg/kg/day, 50 mg/kg/day, 25 mg/kg/day, 12.5 mg/kg/day, or 0 mg/kg/day (for the control group). After 5 weeks, the tumor volumes were measured. FIG. 2 shows that the compound inhibited the tumor growth in a dose-dependent manner. At the dosages of 50 mg/kg/day and 100 mg/kg/day, the tumor volumes decreased.

### Example 9

Male nude mice (6 week old) were transplanted with tumor cells at different body parts. The test compound was orally administered to the mice after certain days of transplantation. The tumor weights were measured and the results are shown in Table 1.

TABLE 1
Inhibition on human tumor transplanted into nude mice

| | | | | Days after | | Inhibition | |
|---|---|---|---|---|---|---|---|
| Transplanted | Tumor | Oral dosage | Test | transplantation | Administration | rate of | |
| tumor | site | (mg/kg/day) | times | (d) | times | tumor (%) | P-value |
| MDA-mb- | chest | 100 | 1 | 16 | 25 | 99 | <0.001 |
| 231 | | 50 | 1 | 16 | 25 | 82 | <0.001 |
| | | 25 | 1 | 16 | 25 | 64 | <0.01 |
| | | 12.5 | 1 | 16 | 25 | 71 | <0.001 |
| SKOV-3 | ovary | 100 | 1 | 18 | 28 | 100 | <0.001 |
| | | 50 | 1 | 18 | 28 | 98 | <0.001 |
| | | 25 | 1 | 18 | 28 | 50 | NS |
| | | 12.5 | 1 | 18 | 28 | 30 | NS |
| LoVo | colon | 100 | 2 | 5 | 14-17 | 99->100 | <0.001 |
| | | 50 | 2 | 5 | 14-17 | 77-81 | <0.01-0.001 |
| | | 25 | 2 | 5 | 14-17 | 55-60 | <0.05-0.001 |
| | | 12.5 | 2 | 5 | 14-17 | 5-27 | NS |
| A549 | lung | 100 | 1 | 14 | 25 | >100 | <0.001 |
| | | 50 | 1 | 14 | 25 | >100 | <0.001 |
| | | 25 | 1 | 14 | 25 | 88 | <0.001 |
| | | 12.5 | 1 | 14 | 25 | 64 | <0.001 |
| | | 12.5 | 1 | 14 | 21-30 | 15-46 | NS-<0.05 |
| A431 | pudendum | 100 | 1 | 14 | 21 | >100 | <0.001 |
| | | 50 | 2 | 14 | 21-30 | 83->100 | <0.001 |
| | | 25 | 2 | 14 | 21-30 | 42-80 | <0.05-<0.001 |
| | | 12.5 | 2 | 14 | 21-30 | 15-46 | NS-<0.05 |

NS = not significant

### Example 10

The test compound as tyrosine kinase inhibitor can inhibit vascular endothelial growth factor receptor (VEGFR) and HUVEC proliferation induced by VEGF, but has no effect on basal cell growth which is not induced by VEGF. Thymidine labeled by ³H is used to measure the cell division of HUVEC in the presence or absence of VEGF, ECF or bFGF.

Thymidine labeled by ³H (10 μCi/mL) and HUVEC (1×10⁵/mL) were co-cultured to allow integration of thymidine into HUVEC. A series of 10-fold dilutions of compound (I) in sterile distilled water were prepared from an initial concentration of 800 mg/L. The dilutions were separately added to into thymidine intergrated HUVEC. The cell division of HUVEC was measured after incubation and IC₅₀ values of the compound for HUVEC were calculated.

As shown in Table 2, the compound significantly and selectively inhibited HUVEC proliferation induced by VEGF, and had no influence on growth of basal endothelial cell even at a concentration 50 times the IC₅₀ for the HUVEC proliferation induced by VEGF. Enzyme analysis shows that the compound exhibited different inhibitory activities against KDR, EGFR and FGFR1 (KDR>EGFR>FGFR1), and cellular composition analysis shows that the compound exhibited different inhibitory activities against VEGF, EGF and bFGF (VEGF>EGF>bFGF). Both analyses suggest that the compound have selective inhibitory activities against various growth factors.

TABLE 2
Inhibition of cell division of basal endothelial cell
and growth factor-induced HUVEC proliferation

| | mean ± error | | | |
|---|---|---|---|---|
| | VEGF | EGF | FGF | Basal |
| IC₅₀(MM) | 0.06 ± 0.02 | 0.16 ± 0.03 | 0.8 ± 0.06 | >3 |
| Experiment | 6 | 6 | 5 | 4 |
| times | | | | |

EGF: epidermal growth factor
FGF: fibroblast growth factor
VEGF: vascular endothelial growth factor

### Example 11

Inhibition of tumor cells by the compound in vitro is evaluated by checking the cell division by use of thymidine labeled by ³H in order to verify whether the compound directly inhibits tumor cell division in vivo as most people believe or indirectly inhibits tumor growth in vivo, for example, by inhibiting angiopoiesis or reducing tumor vasopermeability.

Thymidine labeled by ³H (10 μCi/mL) and tumor cells were co-cultured to allow integration of thymidine into the cells. A series of 10-fold dilutions of the test compound in sterile distilled water were prepared from an initial concentration of 800 mg/L. The dilutions were separately added to into the thymidine intergrated tumor cells. The cell division of the tumor cells was determined after incubation and IC₅₀ values were calculated.

The IC₅₀ values against tumor cell division ranged from 0.8 mm to 1.4 mm (Table 3), 13-230 times the IC₅₀ values against HUVEC division induced by VEGF. This data suggests that the compound inhibit tumor growth by blocking signal factor VEGF of endothelial cell instead of direct inhibiting tumor cell division.

TABLE 3
Influence of the inventive compound on tumor
cell division in vitro (n = 3)

| Tumor cell line | Origin | Mean(±error) IC₅₀ (MM) |
|---|---|---|
| Calu-6 | lung | 1.30 ± 0.05 |
| MDA-MB-231 | Chest | 6.00 ± 1.60 |
| SKOV-3 | ovary | 5.60 ± 0.10 |
| A431 | pudendum | 4.80 ± 0.10 |
| A549 | lung | 3.80 ± 0.40 |
| PC-3 | prostate | 3.70 ± 1.40 |
| LoVo | colon | 0.75 ± 0.20 |

### Example 12

Colon carcinoma LoVo cells were cultured in the logarithmic phase in a 96-well culture plate for 48 h. A compound of this invention in a solution of sterile distilled water at a concentration of 0-100 μg/mL was added to the LoVo cells (6 wells for each concentration). RPMI 1640 culture medium without the compound was used in the control group and the RPMI culture medium without the compound and cells was used in the blank control group. After 72 h, 20 μL MTT (5 g/L) was added into each well. After the culture plate was incubated at 37° C. for 4 h, the culture fluid was removed, 150 μL dimethyl sulfoxide (DMSO) was added to each well, and the absorbance (A) was measured at the wavelength of 570 nm.

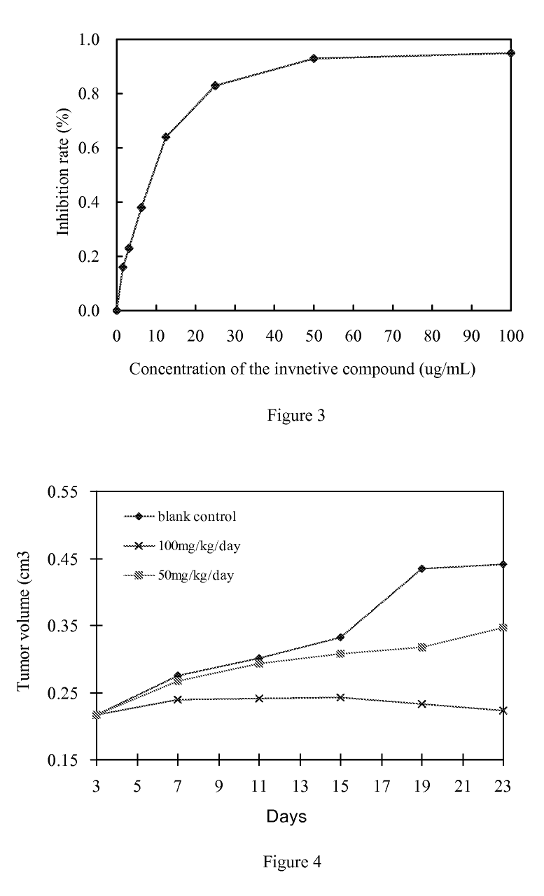

FIG. 3 shows that the compound exhibited inhibitory effect on growth of Lovo cells in a dose-dependent manner. The inhibition rate was 50% when the concentration of the compound is 12.5 μg/mL, and over 90% when the concentration of was 25 μg/mL.

### Example 13

Compound (I) was intraperitoneally injected to a nude mice transplanted with colon carcinoma cell LoVo for 30 days at a dose of 100 mg/kg/day or 50 mg/kg/day. For blank control, 0.2 mL of 0.5% DMSO was intraperitoneally injected to nude mice. The mice were then sacrificed. The tumor volumes were measured for calculating T/C ratio, and weighing tumor for calculating inhibition rate (FIG. 4). The compound selectively inhibited the phosphorylation of KDR tyrosine kinase and blocked the signal transduction of tyrosine kinase, thereby inhibiting the tumor angiogenesis and tumor growth.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the quinazoline compounds of this invention also can be made, screened for their anti-tumor activities, and used to practice this invention. Thus, other embodiments are also within the claims.

What is claimed is:

1. A method of preparing a quinazoline compound of formula (I): 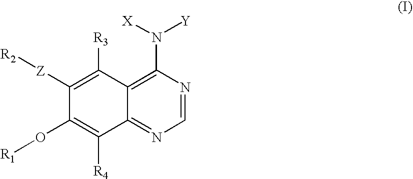
in which X represents —H, methyl, or C₁-C₄ alkyl;
Y represents 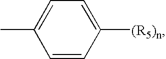
wherein n is 1, 2, 3, or 4, and R₅, independently, is selected from the group of H, methyl, trifluoromethyl, nitro, cyano, C₂-C₄ alkyl, C₂-C₄ alkoxyl, N—(C₂-C₄) alkylamine, enzyme, hydroxyl, N,N-triaza(C₁-C₄) alkylamine, C₁-C₄ alkylthio, and C₁-C₄ alkylsulfonyl;
Z represents 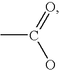
—O, —S, or —NH;
R₁ represents methyl or C₁-C₄ alkyl;
R₂ represents C₁-C₅ alkyl-R₆, C₂-C₆ alkenyl-R₆, or C₂-C₆ alkynyl-R₆, wherein the alkyl, alkenyl, and alkynyl are optionally substituted with one or more akynyl, enzyme, or amino; and R₆ is 4-piperidyl optionally substituted with one or more alkynyl, enzyme, or amino; and
R₃ and R₄, independently, represent H, methyl, C₁-C₄ alkyl, C₂-C₆ alkenyl, C₂-C₆ alkynyl, cycloalkyl, or isocycloalkyl;
the method comprising removing protective group P² from a compound of formula: 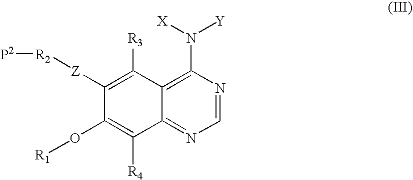
in which X represents H methyl or C₁-C₄ alkyl;
Y represents 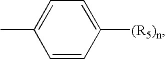
wherein n is 1, 2, 3 or 4, and R₅, independently, is H, methyl, trifluoromethyl, nitro, cyano, C₂-C₄ alkyl, C₂-C₄ alkoxyl, N—(C₂-C₄) alkylamine, enzyme, hydroxyl, N,N-triaza(C₁-C₄) alkylamine, C₁-C₄ alkylthio, or C₁-C₄ alkylsulfonyl;
Z represents —O, —NH, 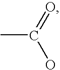
or —S;
R₁ represents methyl or C₁-C₄ alkyl;
R₂ represents C₁-C₅ alkyl-R₆, C₂-C₆ alkenyl-R₆, or C₂-C₆ alkynyl-R₆, wherein the alkyl, alkenyl, and alkynyl are optionally substituted with one or more alkynyl, enzyme, or amino; and R₆ is 4-piperidyl optionally substituted with one or more alkynyl, enzyme, or amino;
R₃ and R₄, independently, represent H, methyl, C₁-C₄ alkyl, C₂-C₆ alkenyl, C₂-C₆ alkynyl, cycloalkyl, or isocycloalkyl; and
P² represents a protective group.

2. The method of claim 1, wherein the compound of formula (III) is as described below:
X represents H;
Y is substituted phenyl, wherein R₅ is alkyl;
Z represents 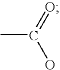
R₁ represents methyl;
R₂ represents C₁-C₅ alkyl-R₆;
R₃ represents C₁-C₄ alkyl;
R₄ represents H; and
P² represents tert-butoxycarboxyl, tert-pentoxycarboxyl, cyclobutoxycarboxyl, propoxycarboxyl, methoxycarboxyl, ethoxycarboxyl, isopropoxycarboxyl, allyloxycarboxyl, or benzyloxycarboxyl.

3. The method of claim 1, wherein the compound of formula (III) is as described below:
X is H;
Y is methylphenyl;
Z is 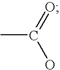
R₁ is methyl;
R₂ is 4-ethylpiperidyl;
R₃ is methyl;
R₄ is H; and
P² is tert-butoxycarboxyl.

4. The method of claim 1, wherein the removing step is performed in the presence of acid.

5. The method of claim 4, wherein the acid is inorganic acid.

6. The method of claim 5, wherein the acid is HCl.

7. The method of claim 1, which the removing step is performed in the presence of an inert solvent and a trace amount of water.

8. The method of claim 7, wherein the inert solvent is dichloromethane or trichloromethane.

9. The method of claim 8, wherein the inert solvent is trichloromethane.

10. The method of claim 1, wherein the removing step is performed at a reaction temperature of 10-100° C.

11. The method of claim 11, wherein the reaction temperature is 20-80° C.

12. The method of claim 4, wherein the acid is organic acid.

13. The method of claim 2, wherein the removing step is performed in the presence of acid.

14. The method of claim 13, wherein the acid is inorganic acid.

15. The method of claim 14, wherein the acid is HCl.

16. The method of claim 2, wherein the removing step is performed in the presence of an inert solvent and a trace amount of water.

17. The method of claim 16, wherein the inert solvent is dichloromethane or trichloromethane.

18. The method of claim 17, wherein the inert solvent is trichloromethane.

19. The method of claim 2, wherein the removing step is performed at a reaction temperature of 10-100° C.

20. The method of claim 19, wherein the reaction temperature is 20-80° C.

21. The method of claim 13, wherein the acid is organic acid.
